(19) United States
(12) Patent Application Publication
(10) Pub. No.: US 2026/0165990 A1
(43) Pub. Date: Jun. 18, 2026

(54) Treatment of Tartaric Acid Toxicity in Dogs with Probenecid

(71) Applicant: MCPHS University, Boston, MA (US)

(72) Inventor: Greg Matthew LANDRY, Boston, MA (US)

(21) Appl. No.: 19/128,654

(22) PCT Filed: Nov. 22, 2023
(86) PCT No.: PCT/US2023/080966

§ 371 Date: May 9, 2025

Related U.S. Application Data
(60) Provisional application No. 63/427,454, filed on Nov. 22, 2022.

(51) Int. Cl. A61K 31/192; A61P 39/02

(52) U.S. Cl. CPC A61K 31/192; A61P 39/02

(57) ABSTRACT

Methods and pharmaceutical compositions for treating and preventing nephrotoxicity of tartaric acid and other organic anions in canines include administering an inhibitor of organic anion transporter-1 (OAT-1) to eliminate toxic accumulation in renal proximal tubule cells due to the absence of organic anion transporter-4 (OAT-4) in canines. Thus, OAT-1 inhibitors such as probenecid can be used to treat or prevent kidney damage from ingestion by dogs of grapes, raisins, or other sources of tartaric acid.

### BACKGROUND

Ingestion of the fruit of Vitis vinifera (common grape) by canines can lead to vomiting, altered mental status, and acute kidney injury¹⁻⁶. Wegenast et. al 2021 recently reported that following ingestion of cream of tartar (KC₄H₅O₆), two dogs experienced vomiting and azotemia. Renal histopathological analysis of one dog revealed striking similarities to that of other canines who had ingested grapes. Therefore, it was proposed that potassium bitartrate, a salt of tartaric acid, could potentially be a canine-specific nephrotoxic compound, as it is also found in significantly high concentrations in grape vacuoles (˜2%; 3.5-11 g/L (19-60 mmol/L)⁸. In canines, ingestion of grapes at doses of 20-150 g/kg (˜196-1484 mg/kg tartaric acid) have been reported to result in significant nephrotoxicity². There is a need for treatments to prevent nephrotoxicity in canines who have ingested grapes or other sources of tartaric acid.

### SUMMARY

The present technology provides compositions and methods to aid in preventing or treating nephrotoxicity in species of animals, such as canines, that are subject to nephrotoxic outcomes after ingestion of tartaric acid. Tartaric acid is a water soluble, common end product of endogenous carboxylic acid metabolism, as well as a well-known component in many dietary fruits that is be primarily filtered, secreted, and excreted unchanged through the kidneys⁸. Tartaric acid, like other carboxylic acids, is understood to be absorbed from the blood into the proximal tubule cell via the basolaterally-localized organic anion transporter-1/3 (OAT-1/3)⁹. In humans and other animals not impacted by ingestion of tartaric acid, when tartaric acid accumulates in proximal tubule cells it is then secreted into the tubular lumen by apically-localized OAT-4 (organic anion transporter 4) for urinary excretion. It has been shown that canines significantly lack expression of renal OAT-4, but do express renally-localized OAT-1¹⁰. This is believed to result in significant proximal tubule cell accumulation of tartaric acid, resulting in canine-specific nephrotoxicity, acute kidney injury, and in many cases, acute renal failure observed following grape or raisin ingestion.

According to the present technology, inhibitors of OAT-1, including probenecid, can be used to prevent or treat nephrotoxicity in dogs resulting from ingestion of tartaric acid or any plant material, such as grapes or raisins, containing tartaric acid. The present technology can be further summarized in the following list of features.
1. A method to aid in treating or preventing nephrotoxicity in a canine subject, the method comprising administering an effective amount of an inhibitor of renal organic anion transporter 1 (OAT-1) to a canine in need thereof, whereby nephrotoxicity in the canine subject is inhibited.
2. The method of feature 1, wherein the inhibitor of OAT-1 is selected from the group consisting of probenecid, amlexanox, telmisartan, mefenamic acid, oxaprozin, parecoxib, sodium meclofenamic acid, nitazoxanide, ketoprofen, ketorolac, tromethamine, and diflunisal.
3. The method of feature 2, wherein the inhibitor of OAT-1 is probenecid or a derivative thereof.
4. The method of feature 3, whereby probenecid is administered to the canine subject at a dose from about 10 mg/kg to about 50 mg/kg, preferably about 25-30 mg/kg.
5. The method of any of the preceding features, wherein the canine subject is a domestic dog, wild dog, wolf, or coyote.
6. The method of any of the preceding features, wherein the canine subject has ingested a substance transported by OAT-1 into renal proximal tubule cells.
7. The method of any of the preceding features, wherein the canine subject has ingested, or is suspected of having ingested, tartaric acid prior to said administration.
8. The method of any of the preceding features, wherein the canine subject has ingested, or is suspected of having ingested, grapes, raisins, apples, cherries, papaya, peach, pear, pineapple, strawberries, mangos, citrus fruits or another fruit or dried fruit product.
9. The method of any of the preceding features, wherein the canine subject has ingested, or is suspected of having ingested a food, a natural substance, a toxin, or a pharmaceutical agent that contains one or more anions that are transported by OAT-1 into renal proximal tubule cells.
10. The method of any of the preceding features, further comprising measuring a blood concentration of tartaric acid in the canine subject before and/or after said administration of said renal OAT-1 inhibitor.
11. The method of feature 10, wherein the measured blood concentration of tartaric acid is used to adjust a dose, frequency, or duration of administration of said renal OAT-1 inhibitor.
12. The method of any of the preceding features, further comprising measuring a blood concentration of said renal OAT-1 inhibitor in the canine subject.
13. The method of any of the preceding features, further comprising measuring a renal function of the canine subject before and/or after said administering of said renal OAT-1 inhibitor.
14. A pharmaceutical dosage form comprising an inhibitor of canine renal OAT-1 and one or more excipients, wherein the inhibitor of canine OAT-1 is present in the pharmaceutical dosage form in an amount effective to inhibit OAT-1 in renal proximal tubules when administered to a canine subject.
15. The pharmaceutical dosage form of feature 14, wherein the OAT-1 inhibitor is selected from the group consisting of probenecid, amlexanox, telmisartan, mefenamic acid, oxaprozin, parecoxib, sodium meclofenamic acid, nitazoxanide, ketoprofen, ketorolac, tromethamine, and diflunisal.
16. The pharmaceutical dosage form of feature 15, wherein the OAT-1 inhibitor is probenecid.
17. The pharmaceutical dosage form of feature 16, wherein the dosage form comprises from about 50 mg probenecid to about 1000 mg probenecid.

### BRIEF DESCRIPTION OF THE DRAWINGS

FIG. 1 shows that tartaric acid induces significant cell death in MDCK cells, but not HK-2 cells, when incubated for 72 h at pH 7.4. Data are represented as mean±SEM (n=3 for the HK-2 cells, n=9 for the MDCK). Asterisks indicate significance when compared to corresponding negative controls (−) as determined by one-way ANOVA followed by a Dunnett's post hoc test, (*) p<0.05; (**) p<0.01; (****) p<0.0001. Additionally, two-way ANOVA was conducted followed by a Bonferroni's post hoc test to compare LDH release of the same tartaric acid concentrations across cell lines. Significant differences were observed between the cell lines.

FIG. 2 shows that probenecid (PBD) at both 0.5 mM and 1 mM concentrations prevents cytotoxicity in MDCK cells induced by 50 mM tartaric acid (TTA)-induced at when incubated for 72 h pH 7.4. Data are represented as mean±SEM (n=3-21). Asterisks indicate significance when compared to corresponding negative controls (−) or 50 mM TTA alone as determined by one-way ANOVA followed by a Tukey's post hoc test. (*) p<0.05; (***) p<0.001; (****) p<0.0001.

FIG. 3 shows the effect of TTA on ATP depletion in MDCK cells as a function of time and dose of TTA exposure. TTA induced significant ATP depletion as shown using a CellTiter Glo® assay, and ATP depletion was both time and dose dependent. TTA concentration groups were compared to media alone (Neg.) using one-way ANOVA followed by a Dunnett's post-hoc test. (n=3/group). * p<0.05; ** p<0.005; *** p<0.001; **** p<0.0001.

FIG. 4 shows the dose dependency of ATP depletion by TTA in MDCK cells at several times of TTA exposure.

FIG. 5 shows that probenecid (PBD) significantly restored ATP levels in MDCK cells in the presence of different concentrations of TTA, at 12 hours and 72 hours exposure to TTA. 500 μM probenecid (PBD) protected MDCK cells from 25 mM and 50 mM TTA-induced ATP depletion at 12 & 72 h, assessed using a CellTiter Glo® assay. Groups were compared using one-way ANOVA followed by a Tukey's post-hoc test. (n=3); ***p<0.001, **** p<0.0001 compared to media; $p<0.05 compared to 25 or 50 mM TTA alone.

FIG. 6 shows that transfection of MDCK cells with human (h) OAT-4 prevents cytotoxicity induced by tartaric acid (TTA, 50 mM). Data are represented as mean±SEM (n=6). Asterisks indicate significance when compared to corresponding controls not transfected with hOAT-4 (−) or the non-transfected group treated with 50 mM TTA alone as determined by one-way ANOVA followed by a Bonferroni's post hoc test. (*) p<0.05; (**) p<0.01; (***) p<0.001, (ns) not significant.

### DETAILED DESCRIPTION

The present technology provides methods of treating and preventing nephrotoxicity of tartaric acid and other organic anions in canines. Tartaric acid is a canine-specific nephrotoxicant found in the common grape. The present inventor has shown that this toxicity in canines is mediated by lack of OAT-4 transporter expression, and accumulation of tartaric acid in the proximal renal tubule. OAT-4 in humans is known to promote the exchange of dicarboxylate anions for other anions at the brush border of proximal tubule cells. The present inventor also has discovered that such toxicity can be averted or treated using probenecid (4-(dipropylsulfamoyl)benzoic acid) or another inhibitor of the OAT-1 transporter. Other suitable OAT-1 inhibitors are described in Duan, P. et al., Mol. Pharm. (2012) 9(11): 3340-3346, which is hereby incorporated by reference in its entirety. Any OAT-1 inhibitor suitable for use in dogs or other canines can be used to prevent or treat nephrotoxicity arising from ingestion of tartaric acid.

The term “canine” as used herein refers to a mammal of the family Canidae, including the genus Canis, and includes all dogs, both domestic and wild, as well as wolves, coyotes, foxes, jackals, and dingoes.

Nephrotoxicity is a graded phenomenon, and as such, “prevention” of nephrotoxicity as used herein refers to any amount of reduction of nephrotoxicity, such as, for example, 5%, 10%, 20%, 30%, 50%, 70%, 80%, 90%, or even 100% reduction in any measure of nephrotoxicity. Nephrotoxicity can be determined by any known measure of renal function, particularly those associated with function of the proximal tubule. For example, plasma markers of renal function can be assessed, such as blood urea nitrogen (BUN) or serum creatinine (sCr). Glomerular filtration rate (eGFR) also can be determined. Urine markers of renal function also can be used, such as urine specific gravity, urine protein level, or urine protein to creatinine ratio. Biopsy also can be used to reveal tissue-level pathology as a measure of nephrotoxicity.

Tartaric acid is dicarboxylic acid found in multiple different fruits and consumer products. Fruits containing tartaric acid include grapes, apples, cherries, papaya, peach, pear, pineapple, strawberries, mangos, and citrus fruits; any of these can cause nephrotoxicity in canines, either in their whole form or in dried form (e.g., raisins). Tartaric acid is known to be synthesized and concentrated significantly in grape vacuoles. It is synthesized from ascorbic acid (Asc) in higher plants through one of three mechanisms classified by the precursor compound and specific cleavage site: Asc C4/C5, Asc C2/C3, and D-gluconic acid C4/C5. In grapes, TTA is predominantly synthesized via the “Asc C4/C5” pathway. Asc is converted via a series of uncharacterized oxidation reactions to 2-keto-L-gluconate, which is then reduced to L-idonate, and subsequently oxidized to 5-keto-D-gluconate¹¹. 5-keto-D-gluconate is the 6-carbon precursor cleaved by tartaric acid semialdehyde and is ultimately oxidized to form TTA¹¹. TTA is then transported and ultimately stored in grape vacuoles via the vacuolar aluminum-activated malate transporter (ALMT9)¹¹. The biologic function of high tartaric acid vacuolar accumulation has not been fully elucidated; however, current hypotheses include its use as a way to reduce oxidative stress in higher plants through decreasing concentrations of ascorbic acid. Additionally, high TTA production in higher plants is a deterrent against predator consumption through the formation of toxic calcium tartrate crystals.

Ingestion of grapes in canines has been shown to cause symptoms ranging from vomiting¹ to acute kidney injury and renal failure². Time of exposure plays a significant role in the development of nephrotoxicity, as canines typically experience severe symptoms after 72 hours if not appropriately treated sooner^{3,4}. As mentioned above, grapes actively accumulate TTA in a range of 3.5-11 g/L (˜19-60 mmol/L)⁸. Furthermore, while ripening, TTA concentrations in grapes can reach as high as 20 g/L (˜100 mmol/L). Therefore, in the in vitro concentration response studies described herein, physiologically relevant TTA concentrations (5, 10, 25, 50, 75, and 100 mmol/L) were utilized.

The results disclosed in the examples below indicate that HK-2 cells show no significant sensitivity to TTA-induced cytotoxicity when compared to controls, as well as no differences when compared to MDCK cells at matching TTA concentrations. However, MDCK cells show significant increases in TTA-induced cytotoxicity compared to controls as low as 10 mmol/L after 72 h of exposure. Therefore, it was found that TTA induces canine specific nephrotoxicity in vitro, and is therefore considered responsible for nephrotoxicity observed in canines post-grape consumption.

Based on endogenous dicarboxylic acid elimination, TTA is thought to be primarily filtered, secreted, and ultimately excreted in the kidney. Thus, renal organic anion transporters (OATs) play a significant role in its accumulation and secretion across the epithelium for ultimate excretion. Canines express only a few, select kidney transporters when compared to other species such as humans, monkeys, mice, and rats. The major kidney transporters detected in canine kidney via quantitative proteomics are the organic anion transporter-1 (OAT-1), organic cation transporter N 2 (OCTN 2 ), multidrug resistance transporter 1 (MDR1), multidrug resistance protein 1 (MRP1), sodium glucose transporter-2 (SGLT-2), and the Na⁺/K⁺-ATPase¹⁰. Treatment with 500 μM and 1 mM probenecid, a known OAT-1 inhibitor, completely prevented TTA-induced cytotoxicity in MDCK cells. These results indicate that preferential OAT-1-mediated transport of TTA in canine kidney cells contributes to TTA-induced nephrotoxic effects in canines.

Basolaterally-localized, renal OAT-1 is expressed across species, as well as in those resistant to grape-induced nephrotoxicity; however, luminally localized renal OAT-4 is species specific.¹⁰ In normal, physiological circumstances renal OAT-4 transports exogenous anions for urinary excretion in exchange for endogenous metabolic intermediate counter ions. In species that lack OAT-4, substances transported by OAT-1 can preferentially accumulate, resulting in renal epithelial damage. OAT-1 is an anion antiporter and belongs to the SLC (solute carrier) family of proteins. It can transport almost any endogenous or exogenous organic anion from the peritubular capillaries transepithelially into the tubular lumen for ultimate excretion. OAT-1 is one of the major secretory transporters, and is a pathway by which non-filtered anions can bypass filtration for urinary excretion. This exchange uses α-ketoglutarate as a counterion. Any organic anion normally secreted through OAT-1 can be accumulated in the blood of an animal treated with an OAT-1 inhibitor. In canines, inhibition of OAT-1 can be used to treat or prevent nephrotoxicity caused by ingestion of tartaric acid or its derivatives, or other organic acids, including some pharmaceutical agents and their derivatives or metabolites, which are excreted in non-canines by OAT-4, or which otherwise accumulate in proximal tubule cells of canines through uptake mediated by OAT-1.

In order to confirm the role of lack of OAT-4 in canine renal proximal tubule cells in TTA-induced nephrotoxicity, MDCK cells were transfected with human OAT-4 and subsequently exposed to a previously determined nephrotoxic TTA concentration (50 mM). Human OAT-4 expression significantly decreased TTA-induced cytotoxicity in MDCK cells indicating a species-specific difference in transporter protein expression as the explanation for grape-induced nephrotoxicity in canines, but not in other species.

As experimentally demonstrated, tartaric acid induced nephrotoxicity occurs through an OAT-mediated mechanism. In canines, the tartaric acid is believed to be absorbed from the peritubular blood supply or reabsorption from the urine via OAT-1, and accumulated in renal cortical tissue with no secretion into the lumen for urinary excretion via OAT-4. Through transfecting MDCK cells with hOAT-4, LDH release (a sign of cytotoxicity) in response to tartaric acid exposure was significantly decreased, giving support to an OAT-4-related mechanism for prevention of TTA-induced nephrotoxicity in non-sensitive species. Furthermore, results from probenecid co-incubation experiments indicate that through inhibiting the absorption of tartaric acid via OAT-1, nephrotoxicity can be decreased or prevented.

In order to treat or prevent nephrotoxicity in canines induced by TTA or other substances accumulated in proximal tubule cells by OAT-1, a pharmaceutical composition containing one or more OAT-1 inhibitors as active ingredients can be administered to a canine subject that has ingested, or is suspected of having ingested, TTA or another OAT-1 accumulated substance. Optionally, the renal function of the canine can be tested before and/or after such administration in order to confirm whether administration of an OAT-1 inhibitor is appropriate, has succeeded in preventing, ameliorating, or eliminating nephrotoxicity, or to adjust the dose or administration schedule of the OAT-1 inhibitor. The pharmaceutical composition can contain one or more excipients in addition to the active ingredient. The dose can be selected so as provide a desired level of inhibition of OAT-1 transport activity and to avoid side effects, such as unwanted accumulation of toxic anions in the blood due to excessive inhibition of OAT-1. For the example of probenecid, in canines an oral dose of 6 mg/kg is enough to completely suppress renal tubular excretion, and this effect appears to plateau at this dose with no further inhibition as the dose is increased. The LD 50 in dogs with probenecid administered IV is 270 mg/kg resulting in increased respiration, muscular twitching, vomiting, micturition and tonic convulsions followed by death due to respiratory arrest within 24 hours. However, this dose is significantly higher than what is be needed to suppress OAT-1 transport for purposes of the present methods. The therapeutic index of probenecid in dogs is high, and toxic concentrations can easily be avoided when treating or preventing grape or raisin intoxicosis.

### EXAMPLE 1

### Materials and Methods

### Cell Culture

MDCK cells were maintained in EMEM media supplemented with 10% Fetal Bovine Serum and 1% streptomycin. Human kidney-2 (HK-2) cells were maintained in DMEM: F12 media supplemented with 10% Fetal Bovine Serum and 1% streptomycin. Both cell lines were incubated at 37° C. and 5% CO₂ until confluency.

For experiments, both cell lines were incubated in triplicate with an endpoint of 72 hours. Treatment concentrations were made using a stock solution of 100 mmol/L potassium bitartrate in complete media. Final treatment concentrations of 5, 10, 25, 50, 75, and 100 mmol/L were reached via serial dilution. To mimic physiological pH of 7.4, 10N NaOH and 10M HCl were used. A positive control of 0.005% hydrogen peroxide was also used.

When conducting experiments with probenecid (Sigma Aldrich, St. Louis, MO), a stock solution of 2 mmol/L in complete media was made at pH 7.4, then mixed with either complete media or 100 mmol/L tartaric acid to yield both solutions of 1 mmol/L and 500 μmol/L and solutions of 1 mmol/L probenecid+50 mmol/L potassium bitartrate, as well as 500 μmol/L+50 mmol/L potassium bitartrate. MDCK cells were treated and allowed to incubate for 72 hours.

hOAT-4 Transfection

In order to assess the role that OAT transporters may play in tartaric acid induced nephrotoxicity, MDCK cells were transfected with human OAT-4 and treated with 50 mmol/L of potassium bitartrate. Human (h)OAT-4 cDNA (GenScript, Piscataway, NJ) was purchased and transformed using E. coli (New England Biolabs, Ipswich, MA) to produce higher amounts of cDNA to be used in transfection. E. coli was transformed per New England Biolab's protocol. Briefly, E. coli was thawed, plasmid cDNA was added, and then mixed with manufacturer's SOC solution. The mixture was allowed to incubate at 37° C. for 60 minutes, and was then spread on LB agar ampicillin-100 plates (Sigma Aldrich, St. Louis, MO) and allowed to incubate for 24 hours at 37° C. Colonies were then collected, placed into a solution of LB agar ampicillin-100 broth, and incubated overnight.

Human OAT-4 plasmid preparation with the amplified E. coli was completed using the PureYield Plasmid Miniprep System (Promega, Madison, WI) following manufacturers protocol. Briefly, 4 mL of culture was collected and centrifuged. The supernatant was removed, and the cell pellet was resuspended in 600 μL 1xPBS. Cell lysis buffer was then added, followed by neutralization solution, and centrifuged. The supernatant was then transferred to a PureYield mini-column, centrifuged, flow through removed, and an endotoxin removal wash solution was added to the column. Finally, elution buffer was added to the minicolumn, solution was centrifuged, and solution of cDNA was produced. The concentration of the elucidated cDNA was then measured using a NanoDrop spectrophotometer (yielding cDNA ranging from 264.8 to 762.8 ng/uL).

MDCK cells were allowed to grow to 70-90% confluency in 24 well plates followed by the addition of 800 ng of elucidated hOAT-4 transporter cDNA with 2 μg of lipofectamine. Cells were then incubated at 37° C. for 2.5 hours. Solution was removed, wells were washed with 1xPBS, and treatment groups as described above were applied to each well. Plates were allowed to incubate for 72 hours, media samples collected for lactate dehydrogenase measurements.

### Measurement of Lactate Dehydrogenase (LDH) Release

Release of lactate dehydrogenase (LDH) from the cytosol in response to plasma membrane damage is a common measure of necrotic cell death and was measured utilizing the LDH-Cytotoxicity assay kit according to the manufacturer's protocol (BioVision, Milpitas, CA). Briefly, media from treated MDCK or HK-2 cells was removed and samples placed into a 96 well plate in duplicate. A mixture of WST kit reagent and buffer in a 25:1 ratio was then added to each well. The plate was covered and allowed to incubate at room temperature for 30 minutes. Absorbance was read at 450 nm. To normalize for potential cell confluency differences, cells were solubilized with 0.05% Triton-X, and cellular protein was analyzed using the bicinchoninic acid (BCA) method.

### Statistical Methods

Data were analyzed via one-way ANOVA followed by Tukey's or Dunnett's post hoc tests to compare differences between individual groups or the negative controls, respectively. Data comparing differences between cell lines at multiple concentrations were analyzed via two-way ANOVA followed by Bonferroni's post hoc test to compare differences between individual species, as well as treatment groups. A significance level of a p<0.05 will be used. All analyses were performed using GraphPad Prism 9. Results are expressed as a mean of multiple experiments±SEM.

### EXAMPLE 2

### Tartaric Acid Cytotoxicity in MDCK Cells

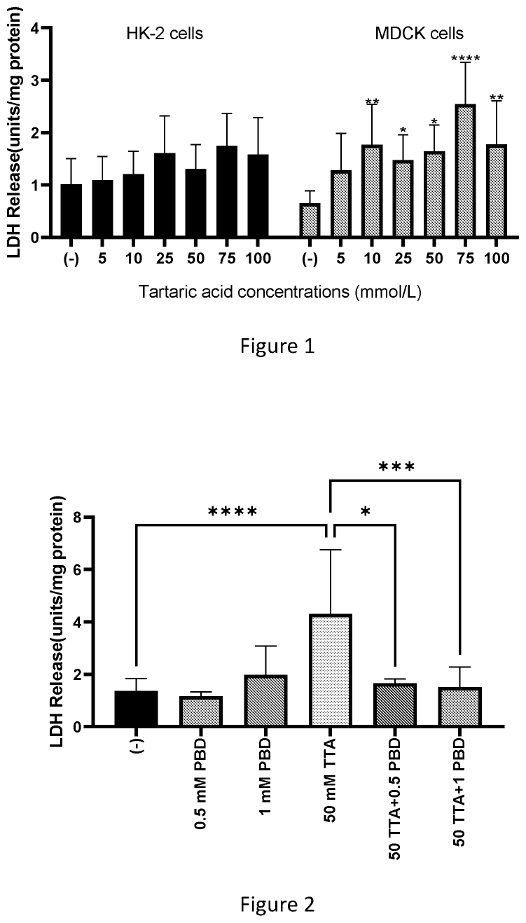

To assess species specific effects of TTA-induced nephrotoxicity, MDCK cells and HK-2 cells were treated for 72 hours with increasing concentrations of potassium bitartrate at pH 7.4. There was no statistically significant increase in LDH release across any of the treatment groups in the HK-2 cells. In the MDCK cells, there was a statistically significant increase in LDH release when comparing the negative controls to the 10 mM (0.6503 vs 1.769, 95% Cl −1.932vs −0.3052, p=0.0032), 25 mM (0.6503 vs 1.472, 95% Cl −1.635-−0.008361, p=0.0468), 50 mM (0.6503 vs 1.646, 95% Cl −1.809vs −0.1827 , p=0.0104), 75 mM (0.6503 vs 2.540, 95% Cl −2.703-−1.077, p<0.0001), and 100 mM (0.6503 vs 1.778, 95% Cl −1.941-−0.3145, p=0.0029) treatment groups (see FIG. 1). Additionally, when comparing HK-2 vs. MDCK cells, there was no significant difference in TTA-induced LDH release at any of the exposure concentrations across species.

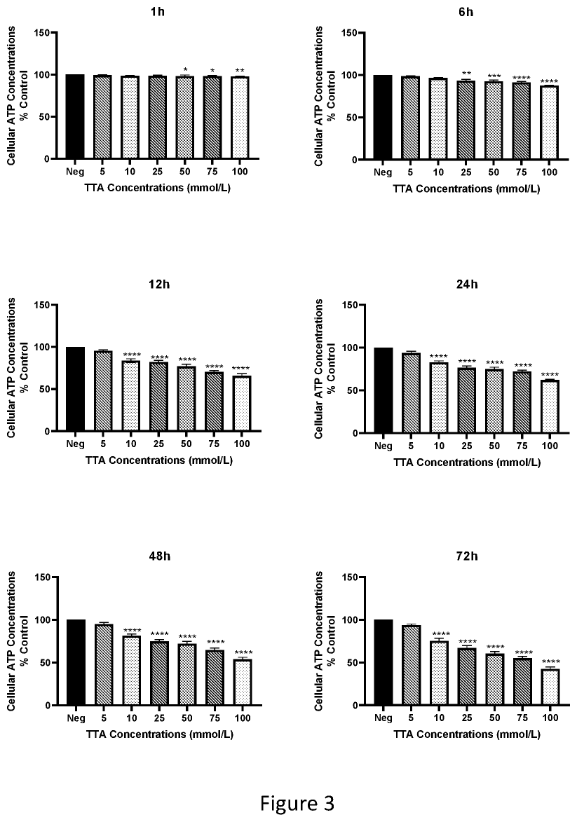

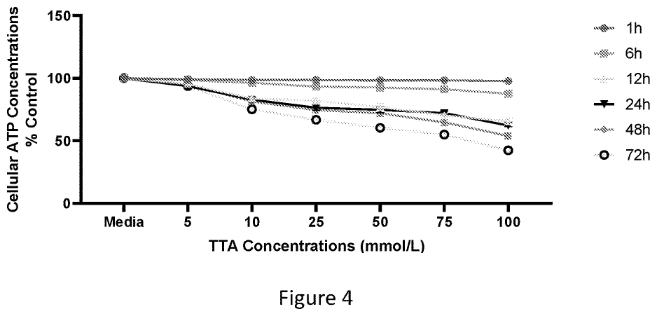

ATP depletion was determined in MDCK cells as another measure of cytotoxicity of TTA. The results shown in FIG. 3 indicate that ATP depletion was both time-and dose-dependent. In FIG. 4, it can be observed that dose-response curves were obtained for TTA-induced ATP depletion, and the cells became more sensitive at each increasing dose, and with increasing time of exposure to TTA. This established the MDCK cell culture model as a valid predictor of TTA-induced renal pathology in canine kidney in vitro.

### EXAMPLE 3

### Probenecid Prevention of Tartaric Acid Induced Cytotoxicity in MDCK Cells

As TTA is a common, dietary dicarboxylic acid, it is believed to be renally excreted via organic anion-handling transporters. Therefore, to assess the role of OAT-mediated TTA transport, MDCK cells were exposed to 50 mM tartaric acid alone or in combination with 1 mM or 0.5 mM probenecid; a known OAT-1 inhibitor. As observed previously, LDH release was significantly increased in the 50 mM TTA treatment group alone when compared to negative control (1.366 vs 4.302, 95% Cl −4.366-−1.506, p<0.0001). Furthermore, 0.5 mM (4.302 vs 1.658, 95% CI 0.2508-5.037, p=0.0226) and 1 mM probenecid (4.302 vs 1.518, 0.8915-4.675, p=0.0009) significantly prevented 50 mM TTA-induced LDH release (see FIG. 2). Additionally, probenecid alone at either concentration did not induce significant cell death in MDCK cells.

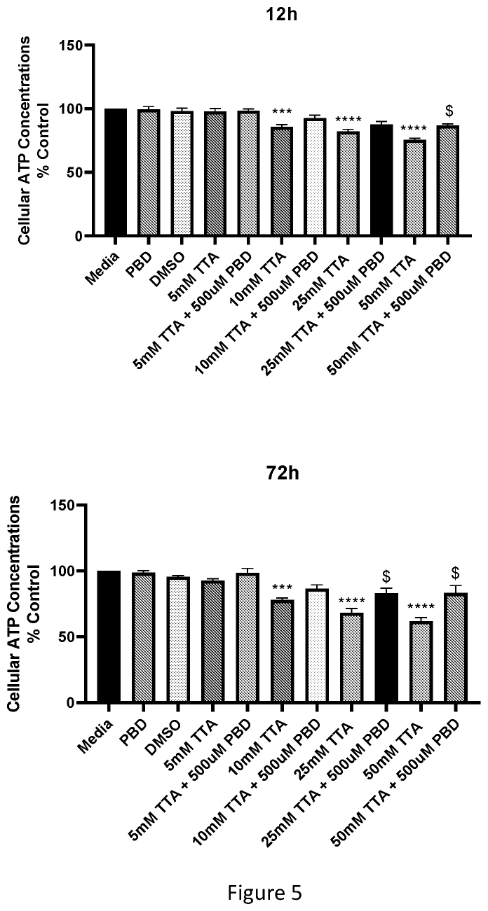

FIG. 5 demonstrates that probenecid can significantly restore ATP levels in TTA-treated MDCK cells, confirming the approach of the present technology as effective in treating or preventing nephrotoxicity in canines that have ingested TTA.

### EXAMPLE 4

### Transfection of MDCK Cells With Human (h)OAT-4

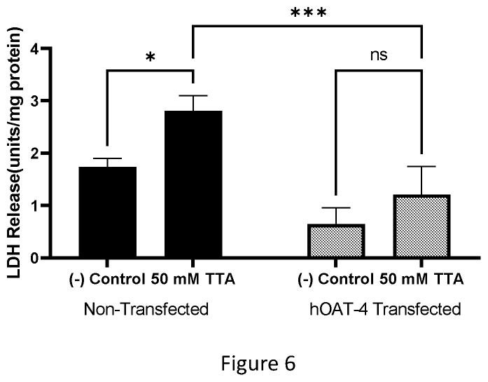

The capability of transfected hOAT-4 in MDCK cells to prevent TTA-induced cytotoxicity was assessed. In non-transfected MDCK cells, there was a significant increase in LDH release in the 50 mM TTA group when compared to the negative control (1.740 vs 2.807, 95% CI of −2.043-−0.09223, p=0.0281). However, in hOAT-4 transfected MDCK cells, there was no significant increase in LDH release in the 50 mM TTA group when compared to the negative control (0.647 vs 1.213, 95% Cl of 1.255-0.1239, p=0.1477). Furthermore, there was a significant decrease in LDH release in the hOAT- 4 transfected MDCK cells exposed to 50 mM TTA when compared to the 50 mM TTA-treated non-transfected cells (2.807 vs 1.213, 95% CI of 0.7495-2.439, p=0.0003) (see FIG. 6).

### REFERENCES

¹Croft R, Clementi E, Farmer H, Whalley R, Dunning M, Firth A. Retrospective evaluation of Vitis vinifera ingestion in dogs presented to emergency clinics in the UK (2012-2016): 606 Cases. J Vet Emerg Crit Care (San Antonio). 2021;31(1):74-79.
²Eubig P A, Brady M S, Gwaltney-Brant S M, Khan S A, Mazzaferro E M, Morrow C M. Acute renal failure in dogs after the ingestion of grapes or raisins: a retrospective evaluation of 43 dogs (1992-2002). J Vet Intern Med. 2005;19(5):663-674.
³Reich C F, Salcedo M C, Koenigshof A M, et al. Retrospective evaluation of the clinical course and outcome following grape or raisin ingestion in dogs (2005-2014): 139 cases. J Vet Emerg Crit Care (San Antonio). 2020;30(1):60-65.
⁴Schweighauser A, Henke D, Oevermann A, Gurtner C, Francey T. Toxicosis with grapes or raisins causing acute kidney injury and neurological signs in dogs. J Vet Intern Med. 2020; 34(5):1957-1966.
⁵Mazzaferro, E. M., Eubig, P. A., Hackett, T. B., Legare, M., Miller, C., Wingfield, W. and Wise, L. (2004), Acute renal failure associated with raisin or grape ingestion in 4 dogs. Journal of Veterinary Emergency and Critical Care, 14: 203-212.
⁶Morrow C M, Valli V E, Volmer P A, Eubig P A. Canine renal pathology associated with grape or raisin ingestion: 10 cases. J Vet Diagn Invest. 2005;17(3):223-231.
⁷Wegenast C, Anderson R, Southard T, Letters to the Editor. 2021 April;258(7). Unique sensitivity of dogs to tartaric acid and implications for toxicity of grapes. Journal of the American Veterinary Medical Association; p.706-707
⁸Moreno J, Peinado R. Grape acids. In: Moreno J, Peinado R, eds. Enological chemistry. London: Academic Press, 2012;121
⁹Nigam S K, Bush K T, Martovetsky G, et al. The organic anion transporter (OAT) family: a systems biology perspective. Physiol Rev. 2015;95(1):83-123
¹⁰Basit A, Radi Z, Vaidya V S, Karasu M, Prasad B. Kidney Cortical Transporter Expression across Species Using Quantitative Proteomics. Drug Metab Dispos. 2019;47(8):802-808.
¹¹Burbidge C A, Ford C M, Melino V J, et al. Biosynthesis and Cellular Functions of Tartaric Acid in Grapevines [published correction appears in Front Plant Sci. 2021 Jul. 9;12:728277]. Front Plant Sci. 2021;12:643024. Published 2021 Mar. 4.

What is claimed is:

1. A method to aid in treating or preventing nephrotoxicity in a canine subject, the method comprising administering an effective amount of an inhibitor of renal organic anion transporter 1 (OAT-1) to a canine in need thereof, whereby nephrotoxicity in the canine subject is inhibited.

2. The method of claim 1, wherein the inhibitor of OAT-1 is selected from the group consisting of probenecid, amlexanox, telmisartan, mefenamic acid, oxaprozin, parecoxib, sodium meclofenamic acid, nitazoxanide, ketoprofen, ketorolac, tromethamine, and diflunisal.

3. The method of claim 2, wherein the inhibitor of OAT-1 is probenecid or a derivative thereof.

4. The method of claim 3, whereby probenecid is administered to the canine subject at a dose from about 10 mg/kg to about 50 mg/kg, preferably about 25-30 mg/kg.

5. The method of claim 1, wherein the canine subject is a domestic dog, wild dog, wolf, or coyote.

6. The method of claim 1, wherein the canine subject has ingested a substance transported by OAT-1 into renal proximal tubule cells.

7. The method of claim 1, wherein the canine subject has ingested, or is suspected of having ingested, tartaric acid prior to said administration.

8. The method of claim 1, wherein the canine subject has ingested, or is suspected of having ingested, grapes, raisins, apples, cherries, papaya, peach, pear, pineapple, strawberries, mangos, citrus fruits or another fruit or dried fruit product.

9. The method of claim 1, wherein the canine subject has ingested, or is suspected of having ingested a food, a natural substance, a toxin, a toxicant, or a pharmaceutical agent that contains one or more anions that are transported by OAT-1 into renal proximal tubule cells.

10. The method of claim 1, further comprising measuring a blood concentration of tartaric acid in the canine subject before and/or after said administration of said renal OAT-1 inhibitor.

11. The method of claim 10, wherein the measured blood concentration of tartaric acid is used to adjust a dose, frequency, or duration of administration of said renal OAT-1 inhibitor.

12. The method of claim 1, further comprising measuring a blood concentration of said renal OAT-1 inhibitor in the canine subject.

13. The method of claim 1, further comprising measuring a renal function of the canine subject before and/or after said administering of said renal OAT-1 inhibitor.

14. A pharmaceutical dosage form comprising an inhibitor of canine renal OAT-1 and one or more excipients, wherein the inhibitor of canine OAT-1 is present in the pharmaceutical dosage form in an amount effective to inhibit OAT-1 in renal proximal tubules when administered to a canine subject.

15. The pharmaceutical dosage form of claim 14, wherein the OAT-1 inhibitor is selected from the group consisting of probenecid, amlexanox, telmisartan, mefenamic acid, oxaprozin, parecoxib, sodium meclofenamic acid, nitazoxanide, ketoprofen, ketorolac, tromethamine, and diflunisal.

16. The pharmaceutical dosage form of claim 15, wherein the OAT-1 inhibitor is probenecid.

17. The pharmaceutical dosage form of claim 16, wherein the dosage form comprises from about 50 mg probenecid to about 1000 mg probenecid.
